(19) United States
(12) Patent Application Publication
(10) Pub. No.: US 2019/0153478 A1
(43) Pub. Date: May 23, 2019

(54) ALKALINE PLANT SALT AND METHOD FOR EXTRACTING THE SAME

(71) Applicant: Sheng Wang, Weifang (CN)

(72) Inventor: Sheng Wang, Weifang (CN)

(21) Appl. No.: 16/168,685

(22) Filed: Oct. 23, 2018

Related U.S. Application Data
(60) Provisional application No. 62/576,789, filed on Oct. 25, 2017.

(51) Int. Cl. C12P 1/00; C12N 9/42

(52) U.S. Cl. CPC C12P 1/00; C12Y 302/01004; C12N 9/2437

(57) ABSTRACT

In one aspect, a method for extracting alkaline plant salt from Suaeda salsa by adopting cellulase and compound protease, comprising enzymolysis and heat leaching. In one embodiment, the enzymolysis may include compound protease enzymolysis, in which an enzymolysis pH value is 6.5-6.8, an enzymolysis time is 4-4.5 hour, and an enzymolysis temperature is 45-55° C. In a further embodiment, in compound protease enzymolysis, the mass ratio of neutral protease and papain is (1.5-2) to 1. In another embodiment, the enzymolysis includes cellulase enzymolysis, an addition quantity of cellulase is 0.15-0.25%, an enzymolysis temperature is 42-47° C., a pH value is 4.5-5, and an enzymolysis time is 4.5-5.5 hour.

### FIELD OF THE INVENTION

The present invention relates to an alkaline plant salt and a method for extracting alkaline plant salt from Suaeda salsa by adopting cellulase and compound protease and belongs to the technical field of plant salt extraction.

### BACKGROUND OF THE INVENTION

It is well known that after a person eats, foods need to be subjected to biochemical process of digestion, decomposition, absorption and metabolism in a digestive system. Nutrients in the foods can be utilized by a human body, and metabolites are excreted to the outsides of the body through sweat glands, urine and feces. A decomposition product of fats is fatty acid, a decomposition product of sugar is lactic acid, and decomposition products of protein are ammonia, uric acid and the like. A pH value of a normal human body fluid is 7.35-7.45 and is weakly alkaline. As the person largely eats foods with high fats, high proteins, high sugar and the like for a long time, a great quantity of acid metabolites are retained in the body; and in addition, as constipation is caused by inadequate dietary fiber intake, toxins are more difficult to excrete, and the human body is in a slight acid sub-health state.

Nutritionists point out that an acidic body can enable metabolism of cells to be weakened and the body resistance to be lowered and cause various diseases. The activity of insulin is lowered by 30% when a pH value of a human body fluid is lowered by 0.1 unit each time, so that diabetes is easily caused, pH values at the peripheries of cancer cells are 6.85-6.95, the acidic body is beneficial to cancer cell survival and metastasis and can cause increase of oxygen free radicals to enable skin to be dry and rough and pigment patches to deposit.

The existing method of extracting the alkaline plant salt from the Suaeda salsa has the following deficiencies:

(1) the prepared plant salt is relatively low in the contents of saponin and alkaloid;

(2) the prepared plant salt is relatively low in the contents of mineral matters, Ca, Mg, P and Zn with an unbalanced ratio;

(3) the prepared plant salt is relatively low in the content of alkaline amino acid; and

(4) the prepared plant salt does not have the improvement action to the acidic body.

### SUMMARY OF THE INVENTION

In order to solve the problems stated above, the present invention provides an alkaline plant salt and a method for extracting alkaline plant salt from Suaeda salsa by adopting cellulase and compound protease to achieve the goals as following:

(1) the plant salt prepared by the present invention contains sodium with the content being 15.20-15.95%;

(2) the plant salt prepared by the present invention contains saponin with the content reaching 0.54-0.66% and alkaloid with the content reaching 2.65-2.78%;

(3) the plant salt prepared by the present invention is rich in the mineral matters and contains Ca with the content reaching 2589-2612 mg/kg, Mg with the content reaching 1.90-1.96%, P with the content reaching 435-440 mg/kg and Zn with the content reaching 76.84-77.65 mg/kg;

(4) the plant salt prepared by the present invention is rich in alkaline amino acid and contains arginine with the content reaching 19.12-19.17 mg/g, lysine with the content reaching 15.48-15.54 mg/g and histidine with the content reaching 1.74-1.79 mg/g; and

(5) the plant salt prepared by the present invention has the action of improving the acidic body.

In order to solve existing problems, the present invention provides an alkaline plant salt, containing the sodium with the content being 15.20-15.95% and the alkaloid with the content reaching 2.65-2.78%. More specifically, the alkaline plant salt contains the Ca with the content reaching 2589-2612 mg/kg.

In one aspect in the present invention, a method for extracting alkaline plant salt from Suaeda salsa by adopting cellulase and compountd protease may include enzymolysis and heat leaching.

In one embodiment, the enzymolysis may include compountd protease enzymolysis; as for the compountd protease enzymolysis, a pH value of the enzymolysis is 6.5-6.8, an enzymolysis time is 4-4.5 h, and an enzymolysis temperature is 45-55° C.

In a further embodiment, in the compountd protease enzymolysis, preferably, a mass ratio of neutral protease and papain is (1.5-2) to 1. In another embodiment, the enzymolysis also comprises cellulase enzymolysis, wherein an addition quantity of cellulase is 0.15-0.25%, an enzymolysis temperature is 42-47° C., a pH value is 4.5-5, and an enzymolysis time is 4.5-5.5 hour.

Heat leaching comprises alkaline leaching and acid leaching. Alkaline leaching may include the following steps: uniformly mixing Suaeda salsa powder and water according to the mass ratio of 1 to (13-17), regulating a pH value to 7.8-8.2, carrying out extraction for 55-65 minutes at a temperature of 53-58° C., carrying out centrifugal separation after extraction, and taking wet residues.

The acid leaching may include the following steps: adding distilled water with the mass being 10-14 times of the mass of wet residues in the wet residues, regulating a pH value to 4.3-4.7, preparing a water-soluble extracting solution, and carrying out leaching; and placing the water-soluble extracting solution in an ultrasonic extractor, wherein an ultrasonic power is 200-240 w, and an extraction time at a temperature of 70-80° C. is 30-40 min.

Compared with conventional techniques, the present invention is advantageous because:

(1) the plant salt prepared by the present invention contains the sodium with the content being 15.20-15.95%;

(2) the plant salt prepared by the present invention contains the saponin with the content reaching 0.54-0.66% and the alkaloid with the content reaching 2.65-2.78%;

(3) the plant salt prepared by the present invention is rich in the mineral matters and contains the Ca with the content reaching 2589-2612 mg/kg, the Mg with the content reaching 1.90-1.96%, the P with the content reaching 435-440 mg/kg and the Zn with the content reaching 76.84-77.65 mg/kg;

(4) the plant salt prepared by the present invention is rich in the alkaline amino acid and contains the arginine with the content reaching 19.12-19.17 mg/g, the lysine with the content reaching 15.48-15.54 mg/g and the histidine with the content reaching 1.74-1.79 mg/g; and

(5) the plant salt prepared by the present invention has the action of improving the acidic body; and after the person eats the plant salt for 3 months, a pH value of blood is increased by 0.8-1.0, the body weight is lost by 1.2-1.5 kg, the defecation times per week are increased by 2.2-2.5 times (being an arithmetic mean value), and a subjectively thought skin color is improved.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

### Embodiment 1

A method for extracting alkaline plant salt from Suaeda salsa by adopting cellulase and compound protease, comprising the following steps:

(1) cleaning:

spreading edible parts of fresh stem leaves and ear axes of the Suaeda salsa with the thickness being 3 cm, spraying dry powder through a nozzle by compressed air which flows at a high speed, utilizing a dry powder-carrying airflow to impact surfaces of plants, and purging off surface attachments, wherein a pressure of the airflow is 0.6-0.7 MPa properly, a purging time is 25-30 min, and an airflow rate is 30-40 kg/L; and

then only using the compressed air to purge the surfaces, and blowing off the dry powder depositing on the surfaces, wherein a pressure of a compressed air flow is 1.3-1.4 MPa, a purging time is 10-15 min, and an airflow rate is 25-30 kg/L;

(2) drying and crushing:

carrying out centrifugal dehydration and drying on cleaned Suaeda salsa, wherein after drying, the moisture content does not exceed 8-10% of the total mass of the Suaeda salsa; and crushing the dried Suaeda salsa into 200 meshes to obtain Suaeda salsa powder;

(3) heat leaching:

A. alkaline leaching:

uniformly mixing the Suaeda salsa powder with water according to the mass ratio of 1 to 15, adding alkaline liquor, regulating a pH value to 8, carrying out extraction for 60 min at the temperature of 55° C., carrying out centrifugal separation after extraction is finished, and taking wet residues; and

B. acid leaching:

adding distilled water with the mass being 12 times of the mass of the wet residues in the wet residues, adding acid, regulating a pH value to 4.5, preparing a water-soluble extracting solution, and placing the water-soluble extracting solution in an ultrasonic extractor, wherein an ultrasonic power is 220 w, and an extraction time at the temperature of 75° C. is 35 min; and after extraction is finished, carrying out centrifugal separation, and taking a supernate;

(4) enzymolysis:

cellulase enzymolysis:

adding cellulase in the supernate obtained in the step (3) for enzymolysis, wherein an addition quantity of the cellulase is 0.2% of the mass of the supernate, an enzymolysis temperature is 45° C., and an enzymolysis time is 5 h; and

compound protease enzymolysis:

regulating a pH value to 6.5, adding compound protease with an addition quantity being 0.8% of the mass of the supernate, and continuously carrying out enzymolysis to obtain an enzymatic hydrolysate, wherein the mass ratio of neutral protease and papain is 2 to 1, a temperature is 50° C., and an enzymolysis time is 4 h;

(5) decoloration:

heating the enzymatic hydrolysate obtained in the step (4) to 55-60° C., dropwise adding acetic acid, regulating a pH value to 6.5-7.0, and adding a flocculant with an addition quantity being 8-10% of the mass of the enzymatic hydrolysate; and starting stirring at the stirring speed of 180 rpm, and continuously stirring; wherein

the flocculant is powder prepared by mixing chitosan, blown seaweed polysaccharide, calcium carbonate and alpha-acid glycoprotein according to the weight ratio being 5 to 2 to 1 to 1; and

the chitosan has the fineness being 100 meshes, the average molecular weight being 1225 Da, the degree of deacetylation being 94.28% and the viscosity being 864 mpa·s;

(6) heavy metal removal:

adding a biological adsorbent with an addition quantity being 0.4-0.6% of the mass of an extracting solution in the extracting solution subjected to decoloration in the step (5), controlling a temperature of the extracting solution at 35-38° C., and stirring for 30-32 h at 100 rpm, wherein

the biological adsorbent is prepared from 10 parts of chitosan, 5 parts of alginate and 3 parts of a microbial preparation,

the microbial preparation is prepared from 1.5×10⁹/g of Saccharomyces cerevisiae, 4.5×10⁸/g of Penicillium chrysogenum, 2.3×10⁸/g of Aureobasidium pullulans and 3.7×10⁸/g of Phanerochaete chrysosporium, microbes are cultured by strains according to a conventional culture method,

a strain preservation number of the Saccharomyces cerevisiae is ACCC20089, a strain preservation number of the Penicillium chrysogenum is ACCC30395, a strain preservation number of the Aureobasidium pullulans is CICC40333, a strain preservation number of the Phanerochaete chrysosporium is CICC40179, and the strains are all commercially available; and

(7) filtering and drying:

filtering a mixture to obtain a Suaeda salsa plant salt filtrate, and carrying out spray drying, wherein as for the spray drying, an air inlet temperature is 140-150° C., an air outlet temperature is 90-100° C., an atomization pressure is 50 mm, a liquid inlet speed is 25%, and an air inlet quantity is 100%.

### Embodiment 2: Combination Factor Analysis Test

By adopting the method described by the Embodiment 1 to extract the plant salt, three factors including an enzymolysis pH value, an enzyme ratio and an enzymolysis time in a compound protease enzymolysis process in the step (4) are changed, and a test is performed.

Table 1 below shows a combination factor analysis test on enzymolysis pH value, enzyme ratio and enzymolysis time in compound protease enzymolysis process; and Table 2 shows indexes of extracted plant salt.

TABLE 1

| | Embod- | Embod- | Embod- | Embod- | Embod- | Embod- |
|---|---|---|---|---|---|---|
| | iment 3 | iment 4 | iment 5 | iment 6 | iment 7 | iment 8 |
| Enzymolysis | 6 | 6.3 | 6.5 | 6.8 | 7.1 | 7.5 |
| pH Value | | | | | | |
| Mass Ratio | 0.5 to 1 | 1 to 1 | 1.5 to 1 | 2 to 1 | 2.5 to 1 | 3 to 1 |
| Of Neutral | | | | | | |
| Protease | | | | | | |
| And Papain | | | | | | |
| Enzymolysis | 3 | 3.5 | 4 | 4.5 | 5 | 5.5 |
| Time | | | | | | |

TABLE 2

| | Embod- | Embod- | Embod- | Embod- | Embod- | Embod- |
|---|---|---|---|---|---|---|
| | iment 3 | iment 4 | iment 5 | iment 6 | iment 7 | iment 8 |
| Sodium (%) | 12.35 | 13.84 | 15.20 | 15.95 | 13.48 | 12.64 |
| Saponin (%) | 0.33 | 0.39 | 0.54 | 0.66 | 0.42 | 0.35 |
| Alkaloid (%) | 2.11 | 2.25 | 2.65 | 2.78 | 2.30 | 2.16 |
| Ca (mg/kg) | 2230 | 2345 | 2589 | 2612 | 2320 | 2280 |
| Mg (%) | 1.62 | 1.73 | 1.90 | 1.96 | 1.78 | 1.65 |
| P (mg/kg) | 410 | 418 | 435 | 440 | 420 | 416 |
| Zn (mg/kg) | 73.25 | 74.34 | 76.84 | 77.65 | 74.95 | 73.54 |
| Arginine | 18.76 | 18.87 | 19.12 | 19.17 | 18.90 | 18.80 |
| (mg/g) | | | | | | |
| Lysine (mg/g) | 15.21 | 15.28 | 15.48 | 15.54 | 15.31 | 15.24 |
| Histidine | 1.50 | 1.57 | 1.74 | 1.79 | 1.60 | 1.55 |
| (mg/g) | | | | | | |

From the factor combination tests, Embodiment 5 and Embodiment 6 are preferred embodiments, i.e. preferably, an enzymolysis pH value in the compound protease enzymolysis process in the step (4) is 6.5-6.8, preferably, a mass ratio of neutral protease and papain is (1.5-2) to 1, and preferably, an enzymolysis time is 4-4.5 hour.

In a further experiment, alkaline plant salts prepared by Embodiment 5 and Embodiment 6 are applied to people with the acidic body and with following characteristics:

people characteristics: the people cannot resist delicious foods, particularly likes to eat sweet foods, frequently participates into social communication, has constipation and is dark in skin color and rough in skin;

people age: 25-50 years old;

people number: 30 persons, two groups with 15 persons per group;

application method: taking 0.5 g in the morning, afternoon and night, respectively, and continuously taking for one month; and

determination of indexes and method: before the alkaline plant salt is taken, a body weight (with an empty stomach at early morning), a pH value of the blood and defecation times per week are counted, and arithmetic mean values are solved respectively; and after the alkaline plant salt is taken for one month, the data is counted again to obtain arithmetic mean values, and shown by taking a difference value obtained by subtracting the two arithmetic mean values from each other as a conclusion, by increasing a subjective judgment of the tester's opinion about his/her own skin color change: the skin color becomes worse (−2), becomes poor (−1), is not changed (0), is improved to some extent (+1), and is obviously improved (+2). Table 3 shows the effect of improving acidic body of plant salt in the present invention.

TABLE 3

| | | | Change Of | |
|---|---|---|---|---|
| | | | Defecation | |
| | | | Times | Subjectively |
| | pH Value | Body Weight | Per Week | Thought Skin |
| | Change | Change (kg) | (Times) | Color Change |
| Embodiment 5 | +0.8 | −1.2 | +2.2 | +1.25 |
| Embodiment 6 | +1.0 | −1.5 | +2.5 | +1.30 |

It is noted that the ratios described by the present invention are all mass ratios, and the percentages described by the present invention are all mass percentages, unless otherwise noted.

Having described the invention by the description and illustrations above, it should be understood that these are exemplary of the invention and are not to be considered as limiting. Accordingly, the invention is not to be considered as limited by the foregoing description, but includes any equivalent.

What is claimed is:

1. An alkaline plant salt comprising sodium for 15.20-15.95%, and alkaloid for 2.65-2.78%.

2. The alkaline plant salt according to claim 1, further comprising Ca for 2589-2612 mg/kg.

3. A method for extracting alkaline plant salt from Suaeda salsa by adopting cellulase and compound protease, comprising enzymolysis and heat leaching.

4. The method for extracting the alkaline plant salt from the Suaeda salsa by adopting the cellulase and the compound protease according to claim 3, wherein the enzymolysis comprises compound protease enzymolysis, in which an enzymolysis pH value is 6.5-6.8, an enzymolysis time is 4-4.5 hour, and an enzymolysis temperature is 45-55° C.

5. The method for extracting the alkaline plant salt from the Suaeda salsa by adopting the cellulase and the compound protease according to claim 4, wherein in compound protease enzymolysis, the mass ratio of neutral protease and papain is (1.5-2) to 1.

6. The method for extracting the alkaline plant salt from the Suaeda salsa by adopting the cellulase and the compound protease according to claim 3, wherein the enzymolysis includes cellulase enzymolysis, an addition quantity of cellulase is 0.15-0.25%, an enzymolysis temperature is 42-47° C., a pH value is 4.5-5, and an enzymolysis time is 4.5-5.5 hour.

7. The method for extracting the alkaline plant salt from the Suaeda salsa by adopting the cellulase and the compound protease according to claim 3, wherein the heat leaching comprises alkaline leaching and acid leaching; and the alkaline leaching comprises the following steps: uniformly mixing Suaeda salsa powder with water according to the mass ratio of 1 to (13-17), regulating a pH value to 7.8-8.2, carrying out extraction for 55-65 minutes at the temperature of 53-58° C., carrying out centrifugal separation after extraction being finished, and taking wet residues.

8. The method for extracting the alkaline plant salt from the Suaeda salsa by adopting the cellulase and the compound protease according to claim 7, wherein the acid leaching comprises the following steps: adding distilled water with the mass being 10-14 times of the mass of the wet residues in the wet residues, regulating a pH value to 4.3-4.7, preparing a water-soluble extracting solution, and carrying out leaching.

9. The method for extracting the alkaline plant salt from the Suaeda salsa by adopting the cellulase and the compound protease according to claim 8, wherein the water-soluble extracting solution is placed in an ultrasonic extractor, an ultrasonic power is 200-240 w, and an extraction time at the temperature of 70-80° C. is 30-40 minutes.
